(19) United States
(12) Patent Application Publication
(10) Pub. No.: US 2015/0361472 A1
(43) Pub. Date: Dec. 17, 2015

(54) AMYLOSE PLASTIC FROM STARCH

(71) Applicant: EMPIRE TECHNOLOGY DEVELOPMENT LLC, Wilmington, DE (US)

(72) Inventor: Manibrata PAUL, Kolkata, West Bengal (IN)

(21) Appl. No.: 14/409,780

(22) PCT Filed: Nov. 20, 2012
(86) PCT No.: PCT/IB12/02397

§ 371 Date: Dec. 19, 2014

(30) Foreign Application Priority Data
Jun. 21, 2012 (IN) 1919/DEL/2012

(51) Int. Cl. C12P 19/16; C08B 33/00; C12P 19/04

(52) U.S. Cl. CPC C12P 19/16; C12P 19/04; C08B 33/00

(57) ABSTRACT

Bioplastics may be produced from starch having an amylopectin content by enzymatically cleaving substantially only α-1,6 bonds of the amylopectin in the starch, leaving the α-1,4 bonds substantially intact. The plastic may be biodegradable, and may be edible if a non-toxic plasticizer is used. Methods for producing the plastic are also disclosed.

### BACKGROUND

A plastic material is generally any of a wide range of synthetic or semi-synthetic organic solids that are moldable. Plastics are typically organic polymers of high molecular mass, but often contain other substances as well. As the quality of plastics improved, the use of plastic expanded rapidly through the 20th century. Plastics are lightweight, flexible, and sturdy and can be used as a replacement for wood, metal and glass. Plastics however, also can have a negative aspect. Toxic chemicals such as benzene and dioxin may be released into surrounding communities during the manufacture of certain types of plastic, and some types of plastics leach chemicals as they are being used. In addition, a plastic bottle tossed into a landfill may take hundreds of years to break down. Plastic bags that litter the landscape may harm animals that try to eat them, and may harm aquatic life when deposited into bodies of water.

Plastic recycling has lightened some of the environmental burden of disposal, but most of the plastics still enter landfills or are incinerated after a single use. In the United States it is estimated that the plastic-bottle recycling rate is less than 25 percent.

Concerns over the environmental impact, health issues, and the rising price and supply petroleum, have encouraged the use and development of bioplastics synthesized from corn, soy, sugar cane, and other crops. Bioplastics are now being used in deli and food containers, and have also been used for automotive parts. Most bioplastic is polymerized lactic acid (PLA). Bioplastics biodegrade relatively quickly under the right conditions, and are made from annually renewable crops rather than petroleum. PLA can also be recycled into more of the same product repeatedly.

One other type of bioplastic is derived from starches. To make plastics from starches, the starch typically requires hydrolysis of cross-linked polysaccharides with a strong acid to produce a usable plastic material. Plastics formed by this process tend to be brittle, lack flexibility and elasticity, are weak in strength, and are not very durable over extended periods of time. However, since starch plastics possess low oxygen permeability, and are digestible and edible, starch plastics are becoming more common. There remains a need for improvements in the quality of starch plastics to make it a replacement for existing non-biodegradable plastics.

### SUMMARY

In an embodiment, a method for producing amylose plastic from amylopectin includes providing amylopectin having cross-linked linear chains of adjacent glucose units bonded together by α-1,4 bonds, with each linear chain being cross-linked to at least one adjacent linear chain by α-1,6 bonds, contacting the amylopectin with at least one enzyme that specifically hydrolyzes substantially only the α-1,6 bonds while substantially leaving the α-1,4 bonds intact, hydrolyzing substantially only the α-1,6 bonds to produce linear amylose, and polymerizing the amylose

In a further embodiment, a method for producing amylose plastic from starch includes providing starch comprising at least about 50% amylopectin having cross-linked linear chains of adjacent glucose units bonded together by α-1,4 bonds, with each linear chain being cross-linked to at least one adjacent linear chain by α-1,6 bonds, contacting the starch and at least one enzyme that specifically hydrolyzes substantially only the α-1,6 bonds while leaving the α-1,4 bonds substantially intact, hydrolyzing substantially only the α-1,6 bonds to produce a mixture of linear amylase wherein a substantial portion of the linear amylose has a length of greater than about 15 glucose units, and polymerizing the amylose.

In an embodiment, a plastic includes polymerized amylose from starch, the starch comprising original amylose and amylopectin, the amylopectin having cross-linked linear amylose chains of adjacent glucose units bonded together by α-1,4 bonds, with each linear amylose chain being cross-linked to at least one adjacent linear amylase chain by α-1,6 bonds, and the polymerized amylase comprises substantially unhydrolyzed original amylose and amylase produced from hydrolysis of substantially only α-1,6 bonds of the amylopectin leaving substantially the α-1,4 bonds intact.

### BRIEF DESCRIPTION OF THE FIGURES

FIG. 1 depicts an illustrative method for producing bioplastic from cross-linked polysaccharides according to an embodiment.

FIG. 2 depicts an illustrative method for producing bioplastic from potato starch according to an embodiment.

### DETAILED DESCRIPTION

Starch is one form of polysaccharide consisting of many glucose units joined together by glycosidic bonds. Starch is essentially made up two types of molecules: long-chain amylase and branched chain amylopectin. Depending on the plant source, starch generally will contain between 10-25% amylose and 75-90% amylopectin. Higher amylose starches are available, pea starch may have about 60% amylase and certain species of maize starch have been developed which may contain up to about 80% amylase.

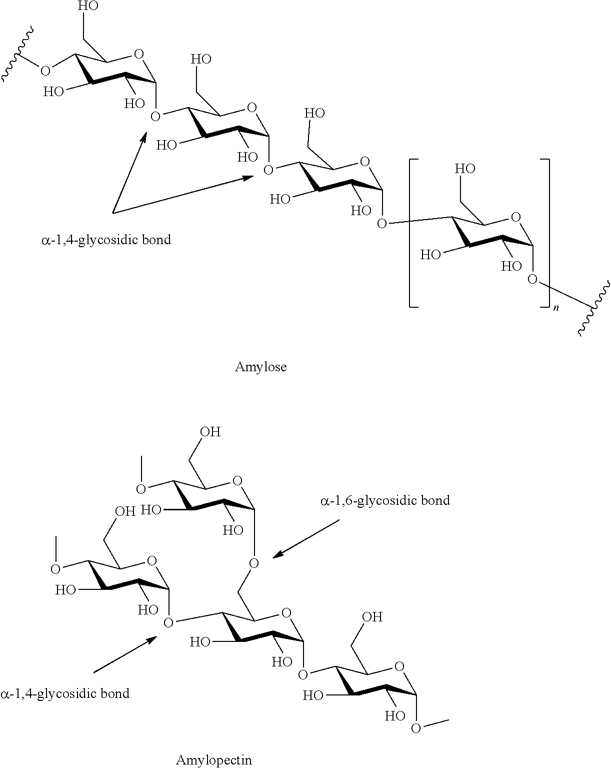

Amylose in starch may typically have at least about 200 glucose units attached together by α-1,4 linkages forming a long chain that will typically have a helical shape because of the bond angles. Amylose may have lengths of up to about 20,000 glucose units. Amylopectin is essentially formed from cross-linked chains of amylose joined together by α-1,6 linkages. The amylose chains in amylopectin are, however, much shorter and average about 20 to about 30 glucose units in length. Amylopectin may contain up to 2 million glucose units.

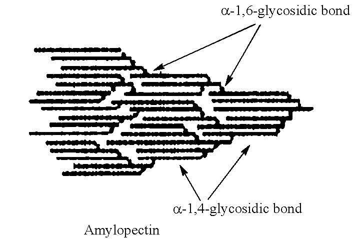

When starch is directly used for manufacturing bio-plastics, with no further processing of the amylose or amylopectin, the resultant plastic is very brittle because of the cross-linking between amylose strands in the amylopectin. One method for improving the plastic quality from starches is to use starch having higher amylose content. Another method is to break up the amylopectin by subjecting the starch to hydrolysis. Hydrolysis may be performed using acid, but acid hydrolysis has several drawbacks. First, acid hydrolysis in non-specific and cleaves individual glucose units off of the amylose and amylopectin, thereby resulting in shorter amylose chains, while also losing a portion of the starch as glucose. Some of the amylopectin remain uncleaved decreasing the quality and value of the polymer. The volume of processing is increased because of the acid and the need for a subsequent neutralization, resulting in a large fluid requirement and making the drying process very energy consuming and time intensive.

Plastics made from cross-linked polysaccharides, such as starch may be achieved over acid hydrolysis by the use of enzymes to break down the cross-linking bonds, wherein for starch, this breaks down the amylopectin into amylose. The quality of the plastics made from starch or any other polymer improves with increasing polymer length, and increased amounts of individual linear polymer strands. The conversion of starch to linear polymers, without substantial loss of polymer length is a key step for producing a better polymer and better yield.

An effective process for producing “amylosic resin” for biodegradable plastic uses the enzymes pectinase, dextranase or pollulanase. A desirable criteria for selection of the enzyme is to use an enzyme which provides substantially only α-1,6 glycosidase/glucanase activity with only minimal or no α-1,4 activity so that substantially only the α-1,6 bonds between the amylose strands of the amylopectin are cleaved with minimal or no chain-shortening cleaving of the α-1,4 bonds. By using α-1,6 hydrolyzing enzymes, essentially all of the amylopectin can be converted into amylase with minimal or no loss of polymer length as glucose monomers. In an embodiment, the enzyme can be chosen so that at least about 97% of the bonds cleaved are α-1,6 bonds and only at most about 3% of the bonds cleaved are α-1,4 bonds. In further embodiments, the percentage of α-1,6 bonds and α-1,4 bonds cleaved should be at least about 98% α-1,6 bonds and at most 2% α-1,4 bonds, or at least about 99% α-1,6 bonds and at most 1% α-1,4 bonds. In an embodiment, it is preferable to select an enzyme with only, or 100% α-1,6 activity with no α-1,4 activity so that only the α-1,6 bonds between the amylose strands of the amylopectin are cleaved with no chain-shortening cleaving of the α-1,4 bonds.

For cross-linked polysaccharides, in an embodiment, the enzyme chosen can be an enzyme which cleaves only the bonds which are holding the cross-linked strands to one another, with minimal to no cleavage of the individual bonds which bond the saccharide units to one another in the individual strands. In this manner, the strand lengths may not be diminished, providing for an improved plastic product.

Starch is one type of polysaccharide which may be usable as a source for bioplastics in accordance with an embodiment. Additional cross-linked polysaccharides which may be usable after a similar cleaving to free individual strands of the polysaccharides may include glycogen, or essentially any polysaccharide which is made up of cross-linked individual chains of molecular units. An appropriate enzyme for cleaving the polysaccharide should be specific for cleaving the cross-linking bonds which bond the individual chains to one another, and have minimal, if any cleavage of the bonds between molecular units forming the individual chains. For glycogen, the enzymes which break down the cross-linking bonds are generally referred to as ‘glycogen debranching enzymes’.

For an embodiment wherein the polysaccharides are starches, the enzymes used can be enzymes which cleave only α-1,6 bonds of amylopectin to convert amylopectin into amylose without cleaving the α-1,4 bonds to break down the amylose into glucose. Enzymes exhibiting α-1,6 specificity include amylopectinase, dextranase, pollulanase, glucanase, and laccase. The use of such enzymes may make it possible to use starches of higher amylopectin content to produce more suitable plastics. Such starches may have amylopectin content of greater than about 50%, or greater than about 55%. or greater than about 60%, or greater than about 65% or greater than about 70%, or greater than about 75%, and such starches may otherwise not be usable for producing plastics if processed by other methods in which hydrolysis of amylase to glucose, and thereby shortening of the amylose chain lengths occurs. Potato starch, for example has about 75% amylopectin and may not be usable for production of suitable plastics if bonds other than α-1,6 bonds are cleaved. Other sources of starch may provide starches of higher amylopectin amounts, which may be up to about 100% amylopectin from glutinous rice, waxy potatoes, and waxy corn.

Plastics produced from the resultant starch in accordance with an embodiment may be biodegradable and edible. These plastics may also exhibit several improvements in their overall properties. Comparison tests on the tensile strength and ultimate elongation of the films showed that the films may display considerably favorable values at 25° C. and relative humidity of 50%, and thus an improvement over acid-hydrolyzed plastics. The changes of properties with the elapse of time, which has also been a problem involving acid-hydrolyzed amylose films, were also studied. After an elapse of one to four weeks, changes in the tensile strength, ultimate elongation, transparency and flexibility for the enzyme hydrolyzed plastics may be minimal. More particularly the plastics prepared in accordance with an embodiment may maintain their original flexibilities even after four weeks. In addition, since the plastics may not display hygroscopicities, the plastics may be durable for prolonged applications.

The plastic produced in accordance with an embodiment wherein substantially only α-1,6 bonds are cleaved in potato starch exhibits the following characteristics: a bulk density of about 1.24 to about 1.31, thermal stability to at least about 275° C. (as measured by a Differential Scanning calorimetry/Thermogravimetric Analyzer (DSC/MA), and an estimated tensile strength of at least about 500 kg/m² or more.

By cleaving substantially only α-1,6 bonds, a substantial portion of the amylase from the hydrolysis of amylopectin should essentially be at least about 10 glucose units in length, and individually hydrolyzed glucose monomers should be minimal, occurring essentially only due to other natural processes, and not the enzymatic hydrolysis. In an embodiment, amylose produced from the hydrolysis of amylopectin has an average or median number of at least about 15 glucose units in length. In an embodiment, at least about 95% of the amylase from the hydrolysis of amylopectin may have a length of at least about 15 glucose units. In another embodiment, at least about 98% of the amylase from the hydrolysis of amylopectin may have a length of at least about 15 glucose units. In another embodiment, about 100% of the amylose from the hydrolysis of amylopectin may have a length of at least about 15 glucose units. For starch having about 25% original amylose content and 75% amylopectin, the resultant mixture after hydrolysis with enzymes can be about 25% long chain amylase (length greater than about 200 glucose units) and about 75% amylase from amylopectin (length greater than about 15 glucose units to a maximum length of about 50 glucose units).

For other polysaccharides, such as glycogen, cleaving substantially only the cross-linking bonds may produce a substantial portion of linear saccharides of at least about 8 glucose units in length, and individually hydrolyzed glucose monomers can be minimal, occurring essentially only due to other natural processes, and not the enzymatic hydrolysis. In an embodiment, at least about 95% of the linear saccharides may have a length of at least about 8 glucose units. In another embodiment, at least about 98% of the linear saccharides may have a length of at least about 8 glucose units. In an embodiment, about 100% of the linear saccharides may have a length of at least about 8 glucose units. In an embodiment, the linear saccharides can have an average or median length of at least about 8 glucose units.

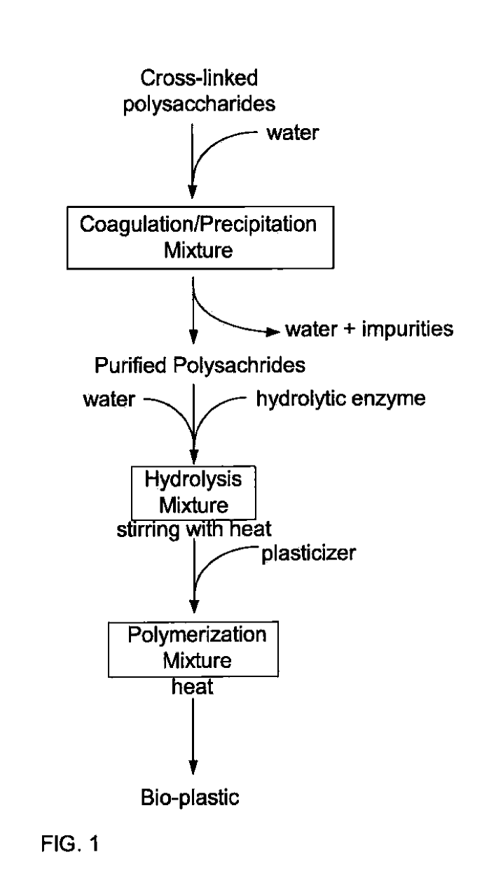

For plastic production from cross-linked polysaccharides, one or several coagulation and precipitation steps may be performed to clean and purify the polysaccharides as illustrated in FIG. 1. This may be done by placing the polysaccharide in an excess of water, followed by stirring, permitting the polysaccharides to settle, and decanting the water from the settled polysaccharides. Once the polysaccharides have been purified, a solution of polysaccharide to water, about 1:2 to about 1:40 by right, may be made and an enzyme selected for its ability to cleave essentially only the bonds holding the cross-linked strands together may be added and the polysaccharide solution may be incubated at a temperature from about 35° C. to about 70° C. for a period of time from about 10 minutes to about 60 minutes or more, if necessary to allow hydrolysis of the cross-linked bonds to occur. The mixture may be vigorously stirred during the heating to enhance hydrolysis. After incubation, the precipitated material may primarily be individual polysaccharide strands, and the polysaccharide strands may be separated from the solution by decanting.

The amount of enzyme added may be selected as a function of amount of polysaccharide, the activity of the enzyme, and the amount of cross-linking present in the polysaccharide. For some enzymes, one enzyme molecule may be capable of hydrolyzing over 1,000,000 bonds. A preferred ratio of enzyme to cross-linked polysaccharide may be determined experimentally for the different enzymes available and the polysaccharide being used. Since enzymes generally do not get consumed, the enzymes may be recovered from solution and reused.

Following separation, the polysaccharides may be mixed with a plasticizer, such as vegetable oil, olive oil or triacetin, or any other compatible plasticizer, and gelatinized to produce the bioplastic materials. Some examples of additional plasticizes which may be used include, but are not limited to polyalcohols (such as glycerol (glycerine), diglycerol, sorbitol, maltitol), choline chloride, tetraethylammonium chloride, N-methylethanolamine, monoethanolamine, tri-ethanolamine 1-, 2- and 3-hexanetriol, ethylene glycol and polyvinylalcohol, alcoholamine. The amount of plasticizer used may be about 1% to about 150% by weight of the polysaccharides, however, in various embodiments an addition of about 5% to about 60% may be preferable. In general, for amounts less than about 5%, the strengths of the plastics formed with the mixture may be improved, but the films may become more brittle. On the other hand, for amounts which exceed about 60%, the elongation strengths of films may be improved, but the films may in some cases lose their strengths and many properties which are undesirable for plastics may become evident.

The polymer binding process (plastic formation) may be initiated by heating the mixture to a temperature of about 70° C. to about 100° C. for a period of time of about 10 minutes to about 30 minutes.

A bioplastic made in this manner may be biodegradable and edible if the plasticizer used is edible, such as vegetable oil or triacetin. In addition, since the enzyme activity is very specific, use of pure enzyme should not contribute to material loss, as occurs with acid hydrolysis based technique where α-1,4 bonds are cleaved and glucose is formed. If potatoes are used to provide starch as the polysaccharide source, wherein there is about 75% amylopectin and 25% amylose in the starch, large portions of the potatoes (almost 75%) in the form of amylopectin may be converted entirely into amylose in order to produce clean and higher yield plastic resins.

Some examples of specific enzymes which may be usable for embodiments of the invention to specifically hydrolyze only α-1,6 bonds of amylopectin include Type I pollulanase enzymes from Aerobacter aerogenes, Fervidobacterium pennavorans Ven5, Bacillus acidopullulyticus, Bacillus flavocaldarius KP 1228, Thermus aquaticus YT-1, Thermals caldophilus GK-24, Thermotoga maritime, and dextranase produced by an oral strain of Actinomyces israelii.

### Example 1

### Production of Plastic from Pectinase-Treated Potato Starch

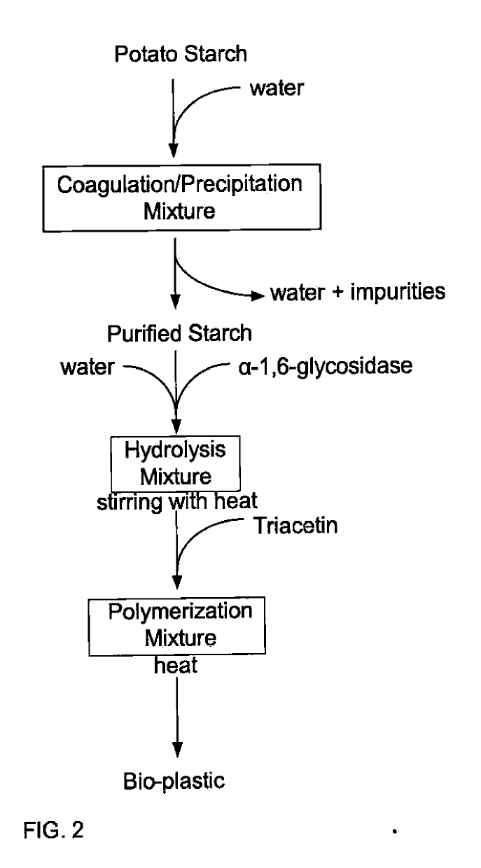

A schematic representation of this process is illustrated in FIG. 2. Potato starch having an amylopectin content of about 75% was cleaned and purified by mixing the starch with water at a ratio of about 1:50 by weight starch to water. The mixture was stirred, allowing the starch to coagulate and then the starch was allowed to precipitate. The coagulating and precipitating were repeated to provide a purified starch.

A mixture was prepared by mixing about 10 g of purified starch in about 400 ml of water in a bioreactor. To this mixture was added about 15 ml of an aqueous preparation of pectinase enzyme from Actinomyces sp., and the mixture was stirred for about 20 minutes at a temperature of about 37° C. to hydrolyze the α-1,6 bonds and break down the amylopectin into individual strands of amylose.

About 5 ml triacetin (plasticizer) was added to the amylose mixture. The mixture was stirred to obtain homogeneity, and then baked in an oven at about 100° C. for about 15 minutes to initiate the polymer binding process

The mixture was spread as a thin film, about 1 mm thick, on a glass surface with a doctor blade and allowed to polymerize into a plastic film.

The prepared film was then analyzed to determine some characteristics. For thermal stability, a thermogravimetric analysis (TGA) was done to determine changes in weight in relation to change in temperature. The film was thermally stable to about 275° C. A bulk density analysis showed that the film had a bulk density of about 1.28 g/cm³. (Additional films prepared with glycerol and refined soy oil as plasticizers showed similar bulk densities of 1.29 g/cm³ and 1.26 g/cm³, respectively.) A tensile strength of the film is expected to be at least about 500 kg/m² or more. The resultant film was sufficiently elastic and stable to make it suitable for plastic wraps, storage bags and containers.

### Example 2

### Production of Plastic from Pectinase-Treated Rice Starch

A schematic representation of this process may also be illustrated in FIG. 2. Rice starch having an amylopectin content of about 85% will be cleaned and purified by mixing the starch with water at a ratio of about 1:50 by weight starch to water. The mixture will be stirred to allow the starch to coagulate and precipitate. The coagulating and precipitating will then be repeated to provide purified starch:

A mixture will be prepared by mixing about 100 g of purified starch in about 500 ml of water in a bioreactor. To this mixture will then be added about 10 ml of pollulanase enzyme preparation, (1.25 g enzyme per ml) and the mixture will be stirred for about 40 minutes at a temperature of about 60° C. to hydrolyze the α-1,6 bonds and break down the amylopectin into individual strands of amylose.

About 25 g glycerol (plasticizer) will be added to the 60° C. amylase mixture. The mixture will be stirred to obtain homogeneity, and then baked in an oven at about 100° C. to initiate the polymer binding process. A film will then be formed on a glass surface and allowed to dry.

### Example 3

### Production of Plastic from Amylase-Treated Potato Starch

In an alternative process for making plastic from potato starch, an enzyme may be used which hydrolyzes amylose and amylopectin with hydrolysis of α-1,4 bonds. Again, with reference to FIG. 2, potato starch having an amylopectin content of about 75% was cleaned and purified by mixing the starch with water at a ratio of about 1:50 by weight starch to water. The mixture was stirred, allowing the starch to coagulate, and the starch was precipitated. The coagulating and precipitating were repeated to provide a purified starch.

A mixture was prepared by mixing about 10 g of purified starch in about 400 ml of water in a bioreactor. To this mixture was added about 15 ml of an aqueous amylase preparation, and the mixture was stirred for about 20 minutes at a temperature of about 37° C. to hydrolyze the starch.

About 5 ml triacetin (plasticizer) was added to the amylose mixture. The mixture was stirred to obtain homogeneity, and then baked in an oven at about 10.0° C. for about 15 minutes to initiate the polymer binding process.

The mixture was spread as a thin film, about 1 mm thick, on a glass surface with a doctor blade and allowed to polymerize into a plastic film.

The resultant film was very brittle, exhibited low elasticity, and was not very stable. The film had a low tensile strength of only about 200 kg/m² and an elongation of only about 12%. As a result, the film had a low crease resistance and tensile strength, and would not be structurally suitable for plastic wraps, storage bags and containers. The film also exhibited some opaqueness, having a light transmission of about 75%, and had a rougher surface texture, both of which qualities, while not affecting the functional properties, would not be as readily acceptable for aesthetic reasons.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the MI scope of equivalents to which such claims are entitled, It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A method for producing amylose plastic from amylopectin, the method comprising:
providing amylopectin, the amylopectin having cross-linked linear chains of adjacent glucose units bonded together by α-1,4 bonds, with each linear chain being cross-linked to at least one adjacent linear chain by α-1,6 bonds;
contacting the amylopectin with at least one enzyme that specifically hydrolyzes substantially only the α-1,6 bonds while substantially leaving the α-1,4 bonds intact;
hydrolyzing substantially only the α-1,6 bonds to produce linear amylose; and
polymerizing the amylose.

2. The method of claim 1, wherein the hydrolyzing produces substantially only linear amylose and at least about 98% of the linear amylose has a length of greater than about 15 glucose units.

3. The method of claim 1, wherein:
the contacting comprises forming an aqueous mixture of the amylopectin and the at least one enzyme; and
the hydrolyzing produces a mixture of linear amylose.

4. The method of claim 1, wherein the amylose plastic has a bulk density of about 1.26 to about 1.29 g/cm³ and a thermal stability of up to about 275° C.

5. (canceled)

6. (canceled)

7. (canceled)

8. The method of claim 1, wherein covalent bonds hydrolyzed by the at least one enzyme comprise at most about 3% α-1,4 bonds.

9. (canceled)

10. The method of claim 1, wherein:
the at least one enzyme is a type I pollulanase, an amylopectinase, a dextranase, or combinations thereof; and
the at least one enzyme hydrolyzes the α-1,6 bonds and does not hydrolyze the α-1,4 bonds.

11. (canceled)

12. (canceled)

13. The method of claim 1, wherein the at least one enzyme is at least one of:
a Type I pollulanase enzyme from Aerobacter aerogenes, Fervidobacterium pennavorans Ven5, Bacillus acidopullulyticus, Bacillus flavocaldarius KP 1228, Thermus aquaticus YT-1, Thermus caldophilus GK-24, and Thermotoga maritime; and
dextranase produced by an oral strain of Actinomyces israelii.

14. The method of claim 1, wherein the polymerizing comprises adding a plasticizer to the mixture of linear amylose for polymerization of the amylose with the plasticizer, wherein the plasticizer is glycerol, triacetin, vegetable oil, olive oil, diglycerol, sorbitol, maltitol, choline chloride, tetraethylammonium chloride, N-methylethanolamine, monoethanolamine, tri-ethanolamine 1-, 2- and 3-hexanetriol, ethylene glycol and polyvinylalcohol, or alcoholamine or any combination thereof.

15. (canceled)

16. A method for producing amylose plastic from starch, the method comprising:
providing starch comprising at least about 50% amylopectin having cross-linked linear chains of adjacent glucose units bonded together by α-1,4 bonds, with each linear chain being cross-linked to at least one adjacent linear chain by α-1,6 bonds;
contacting the starch and at least one enzyme that specifically hydrolyzes substantially only the α-1,6 bonds while leaving the α-1,4 bonds substantially intact;
hydrolyzing substantially only the α-1,6 bonds to produce a mixture of linear amylose wherein a substantial portion of the linear amylose has a length of greater than about 15 glucose units; and
polymerizing the amylose.

17. The method of claim 16, wherein the substantial portion of the linear amylose is at least about 95% of the linear amylose.

18. (canceled)

19. The method of claim 16, wherein:
the starch comprises starch having at least about 70% amylopectin;
the at least one enzyme is an α-1,6-glycosidase which hydrolyzes only α-1,6-glycolytic bonds; and
the at least one enzyme hydrolyzes the α-1,6 bonds and does not hydrolyze the α-1,4 bonds.

20. (canceled)

21. (canceled)

22. The method of claim 16, wherein the at least one enzyme is at least one of:
a Type I pollulanase enzyme from Aerobacter aerogenes, Fervidobacterium pennavorans Ven5, Bacillus acidopullulyticus, Bacillus flavocaldarius KP 1228, Thermus aquaticus YT-1, Thermus caldophilus GK-24, and Thermotoga maritime; and
dextranase produced by an oral strain of Actinomyces israelii.

23. (canceled)

24. (canceled)

25. The method of claim 16, wherein the polymerizing comprises:
adding a plasticizer to the mixture of linear amylose for polymerization of the amylose with the plasticizer; and
heating the mixture of linear amylose and plasticizer to a temperature of about 70° C. to about 100° C. to initiate polymerization of the linear amylose.

26. The method of claim 16, wherein the polymerizing comprises adding glycerol, triacetin, vegetable oil, or olive oil, or any combination thereof to the mixture of linear amylose for polymerization with the amylose.

27. (canceled)

28. (canceled)

29. (canceled)

30. The method of claim 16, wherein:
the forming of the mixture comprises:
mixing starch with water at a ratio of about 1:3 to about 1:5 by weight starch to water; and
adding an effective amount of the at least one enzyme to the starch and water mixture;
the hydrolyzing comprises heating of the enzyme and starch mixture to a temperature of about 35° C. to about 40° C. for about 10 to about 60 minutes while stirring the enzyme and starch mixture; and
the polymerizing comprises:
adding a plasticizer to the amylose mixture in an amount of about 5% to about 60% by weight of the amount of starch; and
heating the mixture of linear amylose and plasticizer to a temperature of about 80° C. to about 100° C. for a period of time of about 25 minutes to about 35 minutes to initiate polymerization of the amylose.

31. (canceled)

32. The method of claim 16, further comprising recovering the at least one enzyme from the aqueous mixture and re-using the at least one enzyme for hydrolyzing additional amylopectin.

33. A plastic comprising polymerized amylose from starch, the starch comprising original amylose and amylopectin, the amylopectin having cross-linked linear amylose chains of adjacent glucose units bonded together by α-1,4 bonds, with each linear amylose chain being cross-linked to at least one adjacent linear amylose chain by α-1,6 bonds, and the polymerized amylose comprises substantially unhydrolyzed original amylose and amylose produced from hydrolysis of substantially only α-1,6 bonds of the amylopectin leaving substantially the α-1,4 bonds intact.

34. The plastic of claim 33, wherein:
the substantially unhydrolyzed original amylose comprises about 98% of the original amylose; and
the amylose produced from hydrolysis of the amylopectin comprises amylose from hydrolysis of at least about 98% α-1,6 bonds and at most about 1% α-1,4 bonds.

35. The plastic of claim 33, wherein the polymerized amylose comprises:
unhydrolyzed original amylose; and
amylose produced from hydrolysis of only α-1,6 bonds of the amylopectin leaving the α-1,4 bonds intact.

36. The plastic of claim 33, wherein the amylose produced from the hydrolysis of the amylopectin consists of amylose having an average chain length of about 15 glucose units.

37. (canceled)

38. The plastic of claim 33, wherein the starch comprises at least about 70% amylopectin and at least about 70% of the polymerized amylose consists of the amylose produced from the hydrolysis of the amylopectin.

39. (canceled)

40. The plastic of claim 33, further comprising a plasticizer polymerized with the amylose, wherein the plasticizer comprises glycerol, triacetin, vegetable oil, olive oil, or combinations thereof.

41. The plastic of claim 33, wherein the plastic has a bulk density of about 1.26 to about 1.29 g/cm³ and a thermal stability of up to about 275° C.
